(19) United States
(12) Patent Application Publication
(10) Pub. No.: US 2009/0264970 A1
(43) Pub. Date: Oct. 22, 2009

(54) Body Heater Suspenders

(75) Inventor: David L. Mickel, Camas, WA (US)

Correspondence Address: Goff Patent Law PLLC, P.O. Box 625, Brush Prairie, WA, 98606, US

(73) Assignee: Jadan Solutions, LLC, Camas, WA (US)

(21) Appl. No.: 12/105,180

(22) Filed: Apr. 17, 2008

(51) Int. Cl. A61F 7/00; A41F 19/00

(52) U.S. Cl. 607/108; 607/112; 604/114; 2/340

(57) ABSTRACT

A portable body heating system includes suspenders with suspender strap(s) adapted to be worn adjacent to a person's torso, and a fastener that is adapted to secure the suspenders an article of clothing covering at least a portion of the person's lower body. The heating system also includes means for securing each of multiple portable heat-producing heat units to one or more of the suspender straps. A method of heating a person's body includes securing suspenders to an article of clothing that covers at least a portion of the person's lower body. The method also includes positioning suspender straps about the person's shoulders, and securing one or more portable heat-producing elements to one or more of the suspender straps. In addition, the method includes positioning an outer clothing layer about the person's body and the heat-producing element(s) so that the outer clothing layer surrounds the heat-producing element(s).

### TECHNICAL FIELD

The description relates generally to heating a person's body, and more particularly to heat units secured to suspenders to heat a person's body.

### BACKGROUND

People have used heat packs, such as chemically-reactive heat packs, to warm their bodies. People typically place such heat packs in gloves or pockets, or otherwise directly secure the heat packs to a fabric layer that encloses a portion of the body where the heat pack is held.

For example, U.S. Patent Publication No. 2007/0106356 by Carstens (the Carstens Publication) discloses “a reusable wrap or tube holder constructed to enclose a portion of the body where the article is to be held.” (See ¶1.) The article that is being held can be a thermal pack. (See Abstract.) While the holder can include “one or more additional straps, panels, or cut-out areas,” (see ¶96), the Carstens Publication notes that the holder is constructed to “enclose a portion of the body where the article is to be held.” (See ¶1).

As another example, U.S. Patent Publication No. 2008/0040831 A1 by Nilforushan et al. (the Nilforushan Publication) discloses securing one or more thermal transfer elements (which can include a heat pack) to an article of clothing. A user wears the article as a layer of clothing, such as an undergarment or an over garment. As in the Carstens Publication, that layer encloses the portion of the body to be heated.

U.S. Pat. No. 5,605,144 to Simmons et al. (the Simmons patent) similarly discloses clothing items that incorporate heat packs. Such clothing items can include scarves or neck wraps that have openings for inserting heat packs. The Simmons patent also discloses a vest to heat the heart and lung area. As with the publications discussed above, these clothing items would enclose the portion of the body to be heated. The Simmons patent states that this design is beneficial because the vest or neck wrap “will provide warmth for the user, thereby enhancing user comfort with or without the heater packets.” (See Col. 6, lines 50-52; Col. 8, lines 24-28).

U.S. Pat. No. 4,061,897 to Thykeson (the Thykeson patent) discloses an electric heating pad that includes belts to hold the heating pad in place. In Thykeson, the heating pad itself covers a large body area to be heated, which can include either “the back area of a person from the lower spine up to and including the shoulders and back of, the neck areas,” or “the front area of a person from the lower abdomen up to and including the chest and shoulder areas.” (Abstract.) Accordingly, it is not surprising that the heat pad described in the Thykeson patent is not portable (i.e., it is not designed to be portable during use). Indeed, the Thykeson patent states that an “object of the instant invention is to provide an electric connection means from the electric heating element in the heating pad for plug-in engagement in a conventional 110V electric receptacle.” (Col. 1, lines 49-52).

### SUMMARY

The present inventor recognized shortcomings of prior body heating apparatuses. For example, in the prior body heating apparatuses discussed above, a heat pack either encloses the portion of the body to be heated or is secured to a layer of material that encloses the portion of the body to be heated. This presents a problem because different types of clothing layers are more appropriate for different types of activities. For example, some types of activities may require clothing layers that retain heat better, while others may require clothing layers that do not retain as much heat, but that breathe better or absorb moisture better. If only one type of clothing layer is used, because the heat packs are secured to that layer, it may not be appropriate for all types of activities. For example, as the Simmons patent notes, its clothing layer will provide warmth to the user even without the heat packs in place. While Simmons presents this as a benefit, the present inventor has recognized it as a problem. For example, in some situations, this may produce too much warmth for the user. Moreover, entire layers of clothing or entire heating pads that enclose portions of a person's body can be expensive, especially when they are made of materials that have good breathability, such as those suggested by both the Carstens Publication (see ¶51) and the Nilforushan Publication (see ¶56).

Other heating apparatuses, such as the one disclosed in the Thykeson patent, are not appropriate for many activities because they are not even designed to be portable. As used herein, the term “portable” refers to a product being portable during use of the product.

Accordingly, there existed a need to provide a body heating apparatus that overcomes one or more of these problems with prior body heating apparatuses. The described embodiments address this need, which has not heretofore been recognized and addressed.

According to one aspect of the described embodiments, a portable body heating system includes suspenders with a set of one or more suspender straps adapted to be worn adjacent to a person's torso, and a fastener that is adapted to secure the suspenders to an article of clothing covering at least a portion of the person's lower body. The heating system also includes means for securing each heat unit of a set of multiple portable heat-producing heat units to one or more of the suspender straps.

The heating system can also include an inner layer of clothing positioned between the suspenders and the person's body, such that the set of heat units is positioned outside the inner layer of clothing. The system can also include an outer layer of clothing positioned outside the suspenders, such that the set of heat units is positioned inside the outer layer of clothing.

In addition, the means for securing can include one or more hook-and-loop fasteners. Also, each heat unit can include a pouch that holds one or more heat-producing elements. The pouch can have metal threads. In addition, the set of heat units can include at least one chemically-reactive heat pack.

According to another aspect of the described embodiments, a system for heating a person's body includes suspenders having a set of one or more suspender straps positioned adjacent to a person's body and secured to an article of clothing, the article of clothing covering at least a portion of the person's lower body. The system also includes a set of one or more portable heat-producing heat units secured to one or more of the suspender straps, and an outer clothing layer surrounding the set of heat units to form a heat envelope around the set of heat units and at least a portion of the person's body.

The system can also include an inner clothing layer positioned between the set of heat units and the person's body. Also, each heat unit can include a pouch and a heat-producing element held in the pouch, and each pouch can be secured to one or more of the suspender straps. The pouch can include fabric with metal threads, and possibly also with non-metal threads. For example, the threads can include nylon and aluminum. The pouches can be secured to the suspender straps with one or more hook and loop fasteners. Also, each heat-producing element of the set of heat-producing elements can include a chemically-reactive heat pack.

According to yet another aspect of the described embodiments, a method of heating a person's body includes securing suspenders, which include a set of one or more suspender straps, to an article of clothing that covers at least a portion of the person's lower body. The method also includes positioning the suspender straps about the person's shoulders, and securing a set of one or more portable heat-producing elements to one or more of the suspender straps. In addition, the method includes positioning an outer clothing layer about the person's body and the set of one or more heat-producing elements so that the outer clothing layer surrounds the set of heat-producing elements.

Positioning the suspender straps can include positioning the suspender straps over an inner layer of clothing, so that the inner layer of clothing is between the person's body and the suspenders. Also, securing the set of one or more heat-producing elements to one or more of the suspender straps can include securing a set of one or more pouches to one or more of the suspender straps, wherein the one or more pouches holds the one or more heat-producing elements. Also, securing the set of one or more heat-producing elements to one or more of the suspender straps can include securing one or more hook-and-loop fasteners.

The various features described herein can be used in combination or independently. Additional features and advantages will be made apparent from the following detailed description of different embodiments that proceeds with reference to the accompanying drawings.

### BRIEF DESCRIPTION OF THE DRAWINGS

FIG. 1 is a front view of a body heating system including body heater suspenders secured to a pair of pants, according to a described embodiment.

FIG. 2 is front view of a body heating system including a pair of body heater suspenders similar to the body heater suspenders of FIG. 1 worn over a person's shirt.

FIG. 3 is a front view of the body heating system of FIG. 2, but additionally including an outer layer of clothing worn over the body heater suspenders, which are shown in dashed lines.

FIG. 4 is a front view of one of the heat units of FIGS. 1-3 secured to a suspender strap.

FIG. 5 is a front view of the heat unit of FIG. 4 with a heat-producing element partially protruding from a pouch as it is being inserted or removed from the pouch.

FIG. 6 is a rear view of the heat unit of FIGS. 4-5.

FIG. 7 is a flowchart showing a use of body heater suspenders according to a described embodiment.

The description and drawings may refer to the same or similar features in different drawings with the same reference numbers.

### DETAILED DESCRIPTION

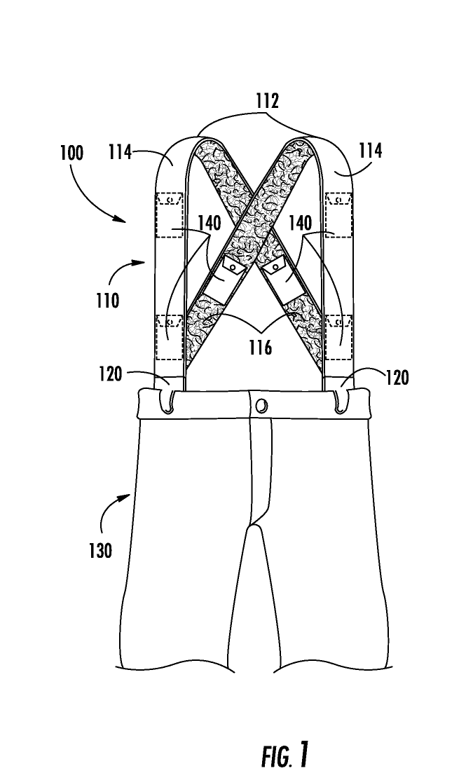

Referring to FIGS. 1-3, a body heating system (100) includes suspenders (110) that include a pair of suspender straps (112) each having an outer surface (114) facing away from a person's body, and an inner surface (116) facing toward the person's body. The suspenders also include suspender fasteners (120) to secure the suspender straps (112) to pants (130), although a person could secure the suspender straps to any article of clothing that covers at least a portion of the person's lower body. As with typical suspenders, the suspender straps (112) extend up from the person's pants (130) along opposite sides of the front of the person's torso, over the person's shoulders, and downwardly in the back to the opposite side of the pants (130). Thus, the suspender straps (112) cross in the back, and they can be used to hold up a person's pants or some other article of clothing that covers a portion of the person's lower body. However, there are many other suspender designs that can be used. The body heating system (100) also includes a set of heat units (140) that can include heat-producing elements, such as those discussed below, to heat the person's body. Each heat unit (140) is removably secured one or both of the suspender straps (112), so that the heat units (140) can be distributed in different positions about the person's torso.

This arrangement produces substantial benefits that are not present in or predictable from prior body heating systems. For example, securing heat-producing elements to suspenders, rather than clothing layers, is beneficial because suspenders typically require less material and can thus be made less expensively than clothing layers. Moreover, many people already use suspenders, and the suspenders (110) can perform the dual functions of holding up a person's pants and carrying the heat units (140) discussed herein. Moreover, suspenders do not produce the breathability problems that can be present with layers of clothing, such as shirts, scarves, or hats. In addition, suspenders can be worn for a wide variety of activities, and the body heating system of FIGS. 1-3 can be worn with a variety of different types of clothing layers, such as t-shirts, button-down shirts, jackets, coats, etc. Thus, the user can choose appropriate clothing layers for the activity to be done by the user. Whatever the chosen clothing layer, the heat produced by the body heating system can be varied by simply attaching or removing heat-producing elements. Indeed, if a person desires to have the body heating system stop producing heat (such as if the person goes indoors), the person can simply remove the heat-producing elements. If that is done, then there is no residing clothing layer (other than the clothes chosen by the user) to keep heating the person when the person does not want to be heated.

Referring still to FIGS. 1-3 and describing the body heating system (100) in more detail, the suspender straps (112) can be standard suspender straps, such as typical 2-inch wide stretchable suspender straps. Alternatively, the suspender straps (112) can be some other width, such as 2¼ inches wide. In one embodiment, each suspender strap is made with a standard 48-inch long strap that is two inches wide and is made of a non-shrinking polyester woven with a common elastic material to allow the strap to stretch. Optionally, each suspender strap (112) can include a common strap adjusting clip (not shown) or other adjustable fastener such as hook-and-loop fastener strips to adjust the length of the suspender strap (112) for different size people. Additionally, the suspender straps (112) can be made of some other material and/or be some other dimensions. For example, shorter straps can be used for children, and longer straps can be used for larger adults.

The outer surface (114) of each suspender strap (112) is formed by the stretchable suspender material discussed above. However, the inner surface (116) of each suspender strap (112) is formed of inwardly-facing loop material, such as the loop material of the hook-and-loop fasteners sold under the name VELCRO by Velcro Industries B.V. and related companies. The loop material can be stretchable so that it will stretch along with the suspender material discussed above. In the illustrated embodiment, the loop material extends along substantially the entire inner surface (116) of each suspender strap (112). Alternatively, the suspenders can include loop tabs or strips spaced apart along the inner surface (116) of the suspender straps. Moreover, the loop material can be on the outer surface (114) of the straps (112) in addition to, or instead of, the loop material on the inner surface (116).

While pants (130) are illustrated in FIGS. 1-3, the suspenders (110) can be secured to other types of clothing articles that cover at least a portion of a person's lower body. For example, the suspenders (110) could be secured to shorts, skirts, or other types of clothing. Indeed, the suspenders (110) could be permanently sewn to a lower-body clothing article, such as with many ski pants or bib overalls. Moreover, the suspenders (110) could be releasably secured to a lower clothing article using fasteners other than the clip fasteners (120). For example, suspenders are commonly secured to pants using buttons, and they could be secured using snaps, hook-and-loop fasteners, or other types of releasable fasteners.

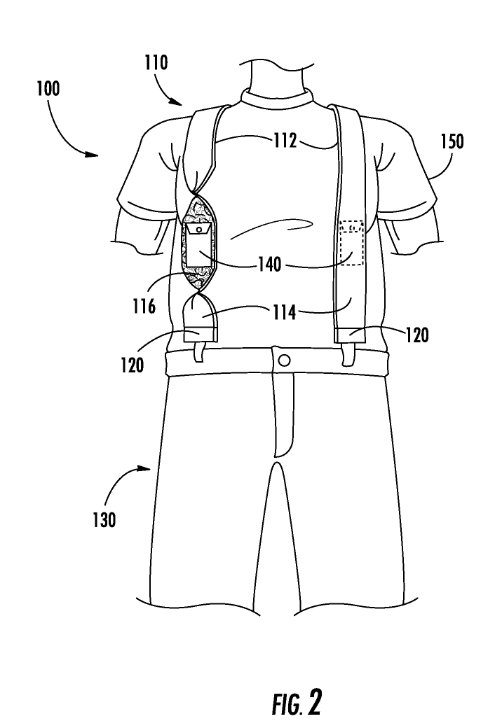

Referring to FIG. 2, the body heating system (100) can further include an inner clothing layer (150). While such an inner clothing layer (150) is not necessary, it is often more comfortable to wear an inner clothing layer, such as a t-shirt or other shirt, under suspenders such as the suspenders (110). It may be desirable to have a different type of inner clothing layer (150) depending on the type of activity that the person is engaging in. For example, it may be desirable for the inner clothing layer (150) to absorb moisture if the activity will result in the person perspiring. In other situations, it may be more desirable for the inner clothing layer (150) to conduct as much heat as possible between the heat units (140) and the person's body, or to disperse the heat to a larger area of a person's body. Because the heat units (140) are secured to the suspenders (110), rather than being secured directly to the inner clothing layer (150), the body heating system (100) can be used with a variety of different types of inner clothing layers, such as short-sleeve shirts, long sleeve shirts, and a variety of material types. In doing so, the person need not modify the inner clothing layer (150) or buy different body heating systems for different activities. The person can simply choose an appropriate inner clothing layer based on the person's likes and dislikes, and based on the type of activity being engaged in, and then use it with the body heating system (100). Or, the person can choose to have no inner clothing layer at all.

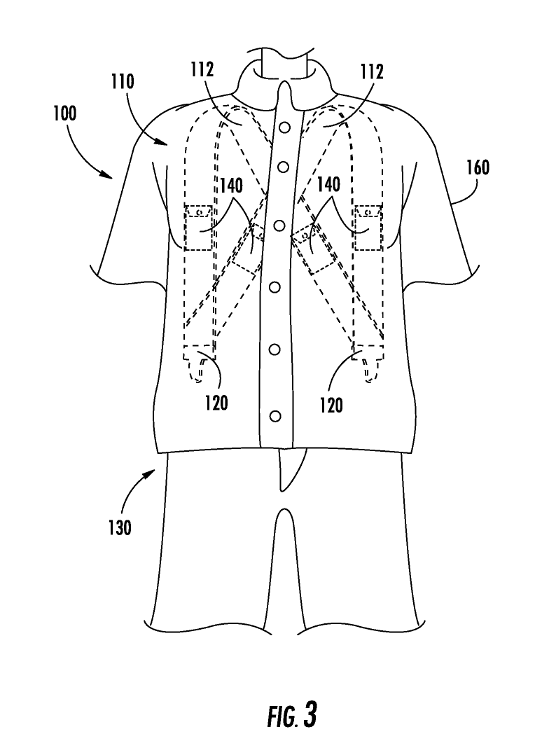

Referring to FIG. 3, the body heating system (100) can also include an outer clothing layer (160). As with the inner clothing layer (150) discussed above, the user can choose an appropriate outer clothing layer (160) based on the type of activity in which the user is engaged. For example, if a person will be in extreme cold weather without engaging in much physical activity, the person can wear a heavy coat or parka as the outer clothing layer (160). In other situations where a person is engaging in physical activities, a lighter jacket, a flannel shirt, or some other type of outer clothing layer may be more appropriate. Whatever the chosen outer clothing layer (160) is, it forms a heat envelope around the set of heat units (140), thereby keeping the heat from the heat units (140) close to the person's body. Thus, the amount of heat transferred from the body heating system (100) to the person's body can be modified by choosing an appropriate outer clothing layer. In addition, once an appropriate outer clothing layer is being worn, the amount of heat can be increased by securing more heat units (140) to the suspender straps (112). Similarly, the amount of heat being transferred to a person's body can easily be decreased by removing one or more heat units (140), or removing one or more heat-producing elements (discussed below) from one or more of the heat pouches (discussed below). Indeed, a person can simple place such removed heat units and/or heat-producing elements in an outer pocket of the outer clothing layer (160) so that they are not within the heat envelope formed by the outer clothing layer (160).

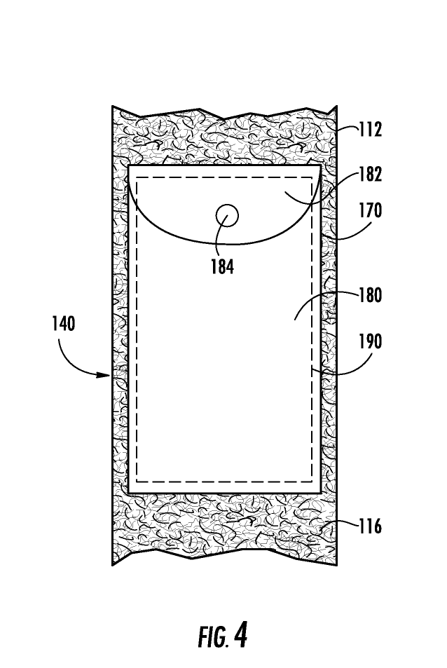

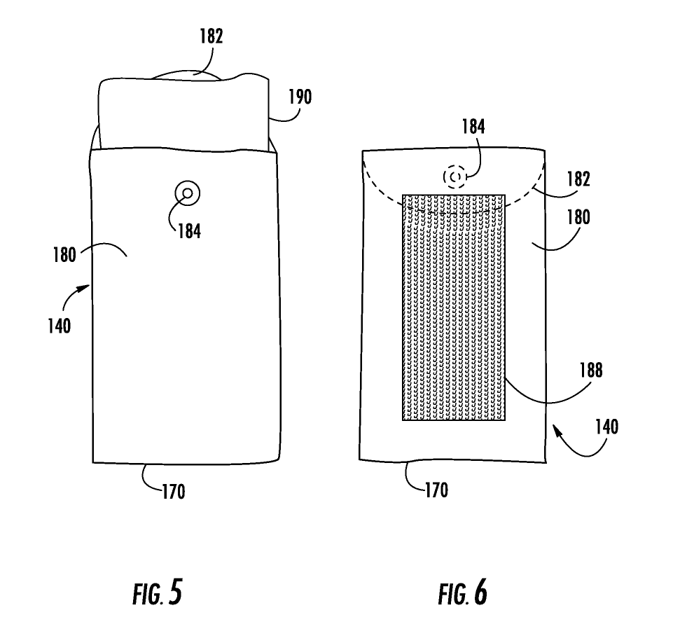

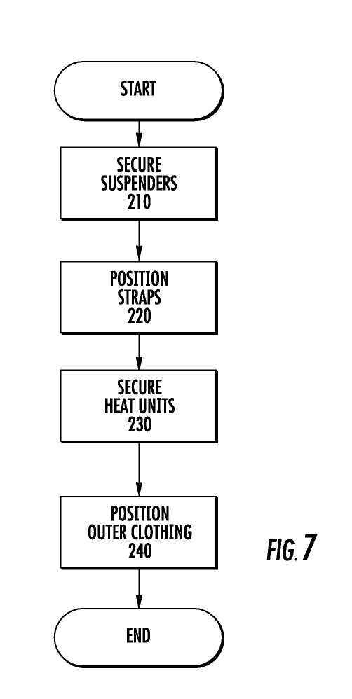

Referring now to FIGS. 4-6, a heat unit (140) includes a heat pouch (170), which defines an enclosing sleeve (180) having an opening therein. The pouch (170) includes a flap (182) that can be secured in a closed position (see FIGS. 4 and 6) with a snap fastener (184). Alternatively, the heat pouch can be held closed with some other type of fastener, such as a button or hook-and-loop fastener. As illustrated in FIG. 6, the heat pouch (170) also includes a hook strip (188). The hook strip (188) can releasably engage the loop material on the inner surface (116) of a suspender strap (112), as shown in FIGS. 1-4, to secure releasably the heat unit (140) to an associated suspender strap (112). Alternatively, each heat unit (140) can be releasably secured to one or both of the suspender straps (112) using some other releasable fastener, such as one or more snaps or buttons. As yet another alternative, each heat unit (140) can be permanently secured to one or more suspender straps (112), such as by sewing the heat pouch (170) in place on the suspender strap(s) (112). Indeed, the heat pouches can be incorporated as pouches within the suspender straps themselves.

The heat pouches (170) can be made of various types of materials, such as fabric materials. It can be advantageous to make the heat pouches of materials that are good conductors of heat so that heat is readily conducted through the pouch and to the person's body. In one embodiment, the heat pouches (170) are made of a woven fabric that is a blend of aluminum and nylon threads. Such a blend can be advantageous because the nylon threads promote flexibility of the fabric, while the aluminum threads are good conductors of heat. The ratio of nylon to aluminum threads can be chosen to achieve desired flexibility and heat conduction properties for particular uses. For example, the fabric could be a ratio of 1:1 aluminum to nylon threads by volume. Alternatively, the ratio could be 1:3 aluminum to nylon threads by volume. However, the fabric could be some other type of polymer and metal blend, or it could be made of polymer or metal threads that are not blended. For example, the pouches could be made of an aluminum thread fabric. The fabric can be sewn together, such as with high-strength nylon thread.

As is illustrated in FIG. 5, each heat unit (140) includes one or more portable heat-producing elements (190), which can be inserted and removed from the heat pouch (170) by releasing the fastener (184) and opening the flap (182) to provide access to the inside of the enclosing sleeve (180). The heat-producing elements (190) can be standard chemically-reactive heat packs, such as those sold under the name LITTLE HOTTIES by Little Hotties Warmers, Inc. of Kent, Wash. Alternatively, the heat-producing elements (190) can be some other type of heat-producing element, such as packs that are pre-heated, such as in a microwave oven, or that are continually heated by a battery power source.

As discussed above and shown in FIGS. 1-4, each heat pouch (170) can be releasably secured to a suspender strap (112). Thus, the loop material on the inner surface (116) of the suspender strap (112) and the hook strips (188) on heat pouches (170), as well as the heat pouches (170) themselves, releasably secure the heat-producing elements to the suspender straps (112) of the suspenders (110) to form the body heating system (100). Accordingly, a person can secure as many heat-producing elements (190) to the suspender straps (112) as the user desires in the positions that the user desires in a particular situation, thereby producing the desired amount and placement of heat by the body heating system (100). As noted above, the body heating system (100) can additionally include one or more inner clothing layers (150) inside the set of heat units (140), as desired. The body heating system (100) can also include one or more outer clothing layers positioned outside the set of heat units (140) to form a heat envelope around at least a portion of the person's body, which can include substantially the entire torso of the person.

Referring now to FIG. 7 and describing the use of body heater suspenders, a person secures (210) suspenders, such as those described above, to an article of clothing, such as a pair of pants, that covers at least a portion of the person's lower body. The person also positions (220) the suspender straps about the person's shoulders, as is typically done with suspenders. This can include positioning the suspender straps over an inner layer of clothing, so that the inner layer of clothing is between the person's body and the suspenders. In addition, the person secures (230) a set of one or more heat-producing elements to one or more of the suspender straps. Securing (230) can include securing one or more hook-and-loop fasteners, such as those described above. Moreover, securing (230) can include securing a set of one or more pouches, which hold the heat-producing elements, to one or more of the suspender straps. Securing can also include securing more heat-producing elements if more heat is desired or removing heat-producing elements if less heat is desired. The person also positions (240) an outer clothing layer about the person's body and the heat-producing elements so that the outer clothing layer surrounds the set of heat-producing elements. Of course, these actions can be performed in different sequences. Moreover, less than all of the actions can be performed in some situations, and additional actions may also be performed by a person using the body heater suspenders.

As can be seen, the body heating system (100) described herein produces many advantageous results, as compared to prior body heating systems. Such results may include one or more of the following: greater portability, decreased expenses, greater variety in the clothing that a user is able to wear with the body heating system, and greater variability in the amount of heat transferred from the body heating system to a person's body.

While the invention has been particularly shown and described with reference to preferred embodiments thereof, it will be understood by those skilled in the art that various changes in form and details may be made therein without departing from the spirit and scope of the invention. For example, the heat-producing units described herein could be secured to one or more of the suspender straps without using a pouch, such as where such a unit is secured to the suspender strap by one or more smaller straps, or in some other manner. As another example, when hook-and-loop material is used to secure the heat-producing units to the suspender straps, the hook material could be on the straps and the loop material could be on the heat-producing units.

I claim:

1. A portable body heating system comprising:
suspenders comprising:
a set of one or more suspender straps adapted to be worn adjacent to a person's torso; and
a fastener that is adapted to engage an article of clothing covering at least a portion of the person's lower body; and
means for securing each heat unit of a set of multiple portable heat-producing heat units to one or more of the suspender straps.

2. The system of claim 1, further comprising an inner layer of clothing positioned between the suspenders and the person's body, such that the set of heat units is positioned outside the inner layer of clothing.

3. The system of claim 1, further comprising an outer layer of clothing positioned outside the suspenders, such that the set of heat units is positioned inside the outer layer of clothing.

4. The system of claim 1, further comprising:
an inner layer of clothing positioned inside the suspenders, such that the set of heat units is positioned outside the inner layer of clothing; and
an outer layer of clothing positioned outside the suspenders, such that the set of heat units is positioned inside the outer layer of clothing.

5. The system of claim 1, wherein the means for securing comprises one or more hook-and-loop fasteners.

6. The system of claim 1, wherein each heat unit comprises a pouch that holds one or more heat-producing elements.

7. The system of claim 6, wherein the pouch comprises metal threads.

8. The system of claim 1, wherein the set of heat units comprises at least one chemically-reactive heat pack.

9. A system for heating a person's body, comprising:
suspenders comprising a set of one or more suspender straps positioned adjacent to a person's body and secured to an article of clothing, the article of clothing covering at least a portion of the person's lower body;
a set of one or more portable heat-producing heat units secured to one or more of the suspender straps; and
an outer clothing layer surrounding the set of heat units to form a heat envelope around the set of heat units and at least a portion of the person's body.

10. The system of claim 9, further comprising an inner clothing layer positioned between the set of heat units and the person's body.

11. The system of claim 9, wherein:
each heat unit of the set of heat units comprises a pouch and a heat-producing element held in the pouch; and
each pouch is secured to one or more of the suspender straps.

12. The system of claim 11, wherein each pouch comprises a fabric that comprises metal threads.

13. The system of claim 12, wherein the fabric comprises metal threads and non-metal threads.

14. The system of claim 13, wherein the non-metal threads comprise nylon and the metal threads comprise aluminum.

15. The system of claim 11, wherein each pouch of the set of pouches is secured to one or more of the suspender straps by one or more hook-and-loop fasteners.

16. The system of claim 9, wherein each heat-producing element of the set of heat-producing elements comprises a chemically-reactive heat pack.

17. A method of heating a person's body comprising:
securing suspenders comprising a set of one or more suspender straps to an article of clothing that covers at least a portion of the person's lower body;
positioning the suspender straps about the person's shoulders;
securing a set of one or more portable heat-producing elements to one or more of the suspender straps; and
positioning an outer clothing layer about the person's body and the set of one or more heat-producing elements so that the outer clothing layer surrounds the set of heat-producing elements.

18. The method of claim 17, wherein positioning the suspender straps comprises positioning the suspender straps over an inner layer of clothing, so that the inner layer of clothing is between the person's body and the suspenders.

19. The method of claim 17, wherein securing the set of one or more heat-producing elements to one or more of the suspender straps comprises securing a set of one or more pouches to one or more of the suspender straps, wherein the one or more pouches holds the one or more heat-producing elements.

20. The method of claim 19, wherein securing the set of one or more heat-producing elements to one or more of the suspender straps comprises securing one or more hook-and-loop fasteners.
